(19) United States
(12) Patent Application Publication
(10) Pub. No.: US 2011/0264043 A1
(43) Pub. Date: Oct. 27, 2011

(54) METHOD OF LOADING A DRUG LIBRARY INTO AN INFUSION PUMP

(75) Inventors: Paul T. Kotnik, Commerce Township, MI (US); Troy A. Bartz, Lake Orion, MI (US)

(73) Assignee: CURLIN MEDICAL INC., East Aurora, NY (US)

(21) Appl. No.: 13/127,506

(22) PCT Filed: Oct. 22, 2009
(86) PCT No.: PCT/US09/61579

§ 371 Date: Jul. 13, 2011

Related U.S. Application Data
(60) Provisional application No. 61/198,683, filed on Nov. 7, 2008.

(51) Int. Cl. A61M 5/142; H04W 4/00; A61P 29/00; A61K 31/192; A61K 31/167

(52) U.S. Cl. 604/151; 514/570; 514/629; 455/41.2

(57) ABSTRACT

A method of loading a drug library into an infusion pump includes transferring the drug library, stored on a remote device, to the infusion pump via a wireless connection. The drug library includes a plurality of subdirectories, and each subdirectory includes a care area, at least one drug, data associated with and dependent upon the drug(s), and at least one mode of operation for infusing the drug(s).

### CROSS REFERENCE TO RELATED APPLICATION

This application claims the benefit of U.S. Provisional Patent Application Ser. No. 61/198,683 for a METHOD OF LOADING A DRUG LIBRARY INTO AN INFUSION PUMP, filed on Nov. 7, 2008, which is hereby incorporated by reference in its entirety. This claim is made under 35 U.S.C. §119(e); 37 C.F.R. §1.78; and 65 Fed. Reg. 50093.

### TECHNICAL FIELD OF INVENTION

The present disclosure relates generally to infusion pumps and, more particularly, to a method of loading a drug library into an infusion pump.

### BACKGROUND

Infusion pumps are often used to deliver fluid in a controlled manner such as, for example, an intravenous delivery of pharmaceutical compositions (i.e., a drug) to a patient or subject. In many cases, the infusion pump is programmable with an infusion protocol for delivering the drug to the patient. The infusion protocol, which may, for example, include a name of a drug, a dosage of the drug, and a mode of delivering the drug to the patient, is checked against pre-established safety standards. These pre-established safety standards are often retrieved from a master drug library.

### SUMMARY

A method of loading a drug library into an infusion pump includes transferring the drug library, stored on a remote device, to the infusion pump via a wireless connection. The drug library includes a plurality of subdirectories, each of which includes a care area, at least one drug, data associated with and dependent upon the drug(s), and at least one mode of operation for infusing the drug(s).

### BRIEF DESCRIPTION OF THE DRAWINGS

Features and advantages of embodiment(s) of the present disclosure will become apparent by reference to the following detailed description and drawings, in which like reference numerals correspond to similar, though perhaps not identical components. Reference numerals having a previously described function may or may not be described in connection with other drawings in which they appear.

FIG. 1 is a semi-schematic depiction of an example of an infusion pump system;

FIG. 2 is an example of a master drug library; and

FIG. 3 is a flow diagram depicting an example of a method of loading a drug library into an infusion pump.

### DETAILED DESCRIPTION

Embodiment(s) of the method disclosed herein are advantageously used to quickly, easily, and reliably transfer a drug library from a remotely-located device (e.g., a computer) to an infusion pump. The drug library may be used by the infusion pump to verify that parameters included in an infusion protocol for infusing a drug fall within accepted safety standards. Further, the entire drug library is transferred to the infusion pump, which at least allows the infusion pump to run a number of different infusions without having to retrieve new part(s) of the drug library from the remote device for each separate infusion.

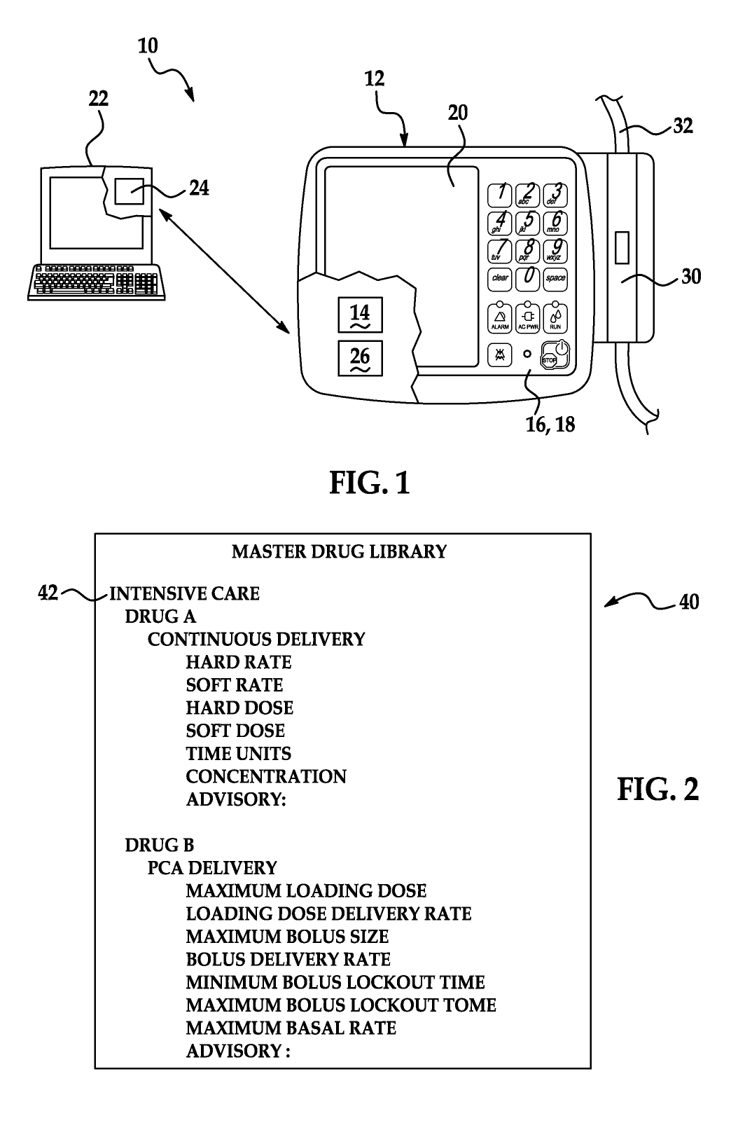

An example of an infusion pump system 10 that may be used for embodiment(s) of the method disclosed herein is semi-schematically depicted in FIG. 1. The infusion pump system 10 includes an infusion pump 12, such as a rotary peristaltic pump as shown in FIG. 1. Such rotary peristaltic infusion pumps may include a removable cassette 30 including an assembly of rollers (not shown) and a flexible tube 32 that surrounds a portion of the assembly of rollers. In response to rotational movement of the rollers, portions of the flexible tube 32 in contact with the rollers compress or otherwise occlude against a wall of the cassette 30. As a result, fluid (i.e., a drug) traveling through the tube 32 is temporarily trapped in the tube 32 between the occluded points. The trapped drug is released from the tube 32 when the occlusion force on the tube 32 is released. In this manner, the drug is urged through the tube 32 via peristaltic wave action and is ultimately delivered to a patient or subject.

While a rotary peristaltic pump is shown and described herein, it is to be understood that other infusion pumps are also suitable for use in the methods of the present disclosure. Examples of such other infusion pumps include syringe pumps and linear peristaltic pumps.

The infusion pump 12 also includes a user interface 16 operatively connected thereto. In an embodiment, as shown in FIG. 1, the user interface 16 is a twelve-digit keypad 18, operatively associated with a display screen 20. Together, the interface 16 and display screen 20 enable manual entry of, e.g., drug-related data into the infusion pump 12. The display screen 20 may, in an example, be a standard display exhibiting black-and-white and/or color graphic and alpha-numeric characters. The display screen 20 may, in another example, be a color touch screen.

In some instances, the display screen 20 may further include an ambient light detection feature (not shown) to determine how optically bright the ambient environment is. The light detection feature may automatically adjust the backlighting of the display screen 20 depending on increased or decreased lighting of the ambient environment. Further, the display screen 20 may include a dimming feature, where the optical brightness of the display screen 20 dims when the infusion pump 12 is continuously operated at substantially the same setting(s).

In another embodiment, the user interface 16 is a machine-readable scanner (not shown) operatively associated with the infusion pump 12. The machine-readable scanner wirelessly transmits information to the infusion pump 12 via a wireless connection established between the scanner and the pump 12. In an example, the machine-readable scanner is a barcode scanner configured to read barcode labels having information stored thereon related to, e.g., a drug to be infused, data associated with the drug, and a protocol for infusing the drug. Such information may be used to program the infusion pump 12 as described in further detail in U.S. Patent Provisional Application Ser. No. 61/198,681 (Docket No. DP-318233), the disclosure of which is herein incorporated by reference in its entirety.

The infusion pump 12 also has associated therewith a memory 14 for storing a drug library wirelessly transmitted thereto from a remote device 22. The transmission of the drug library to the infusion pump 12 may be enabled using a wireless connection established between suitable hardware 24 of the computer and complementary hardware 26 of the infusion pump 12. Non-limiting examples of wireless connections include Wi-Fi, Bluetooth®, Zigbee®, wireless Ethernet, and/or like, and/or combinations thereof. The wireless transfer or transmission of the drug library from the remote device 22 to the infusion pump 12 advantageously allows the transfer without having to wire the device 22 to the pump 12 and without having to move the remote device 22 along with the infusion pump 12 from one location to another.

The remote device 22 is shown in FIG. 1 as a computer. It is to be understood, however, that other remote devices such as, e.g., a personal digital assistant or a cellular phone may also be used.

An example of a master drug library 40 is generally depicted in FIG. 2. The drug library 40 generally includes a plurality of subdirectories 42, each of which is associated with one or more different drugs. In the example shown in FIG. 2, the master drug library 40 includes one subdirectory 42: Intensive Care. Each subdirectory 42 is labeled with a care area, drug(s) (e.g., Drugs A and B) that will be used, a mode of operation for infusing the drug(s), and data associated with the drug(s) to be infused.

The care area corresponds to an area of a medical facility where the infusion will take place. Non-limiting examples of care areas include an intensive care unit (ICU), pediatrics, burn unit, trauma unit, etc.

Listed under each care area is at least one drug (e.g., Drug A and Drug B as shown in FIG. 2). The drug is generally identified by its drug name such as, e.g., its chemical name based on the main ingredient of the drug or the trademarked names of the drug. For example, if the drug name is provided as “Ibuprofen,” the trademarked names “Advil®” or Motrin® may be listed below it as subcategories. In another example, the drug name may be provided using only the trademarked name of the drug. Using the example shown in FIG. 2, the drug name for “Drug A” may be, e.g., “Advil®’” and for “Drug B,” the drug name may be Tylenol®.

The modes of operation correspond to the type of drug delivery that may be used to deliver the drug to the patient using the infusion pump 12. An appropriate mode of operation is provided under each drug name. In many instances, the mode of operation is dependent on the care area that the infusion pump 12 will be used in. Non-limiting examples of the modes of operation include a continuous delivery, a weight-based continuous delivery, an intermittent delivery, a patient controlled analgesia delivery, a dose-based delivery, a total parenteral nutrition delivery, and/or the like.

Data associated with and dependent upon the drug includes any data that corresponds to predetermined limits of infusing the drug that generally fall within safety standards for a single dose of the drug. These limits (also referred to herein as infusion limits) may include, for example, a concentration of the drug to be infused, hard and/or soft rates of delivering the drug, hard and/or soft dosage limits of the drug, a listing of possible side effects from the drug (included, for example, in an advisory section listed under each subdirectory 42), and/or the like. It is to be understood that the data associated with the drug included in each subdirectory 42 is dependent upon, at least in part, the mode of operation for infusing the drug. The drug data listed in each subdirectory 42 for each mode of operation is provided below:

For continuous delivery, the data or limits included in each subdirectory 42 include a hard rate limit, a soft rate limit, a hard dose limit (e.g., hard dose rate limit and hard dose vtbi limit), a soft dose limit (e.g., soft dose rate limit and soft dose vtbi limit), units for time, a concentration of the drug, units for the concentration, advisory information, or combinations thereof.

For weight-based continuous delivery, the limits included in each subdirectory 42 include a hard rate limit, a soft rate limit, a hard dose limit (e.g., hard dose rate limit and hard dose vtbi limit), a soft dose limit (e.g., soft dose rate limit and soft dose vtbi limit), units for time, a concentration of the drug, units for the concentration, advisory information, or combinations thereof.

For delivery based on a dose, the limits included in each subdirectory 42 include a hard rate limit, a soft rate limit, a hard dose limit (e.g., hard dose rate limit and hard dose vtbi limit), a soft dose limit (e.g., soft dose rate limit and soft dose vtbi limit), units for time, a minimum delay time, a concentration of the drug, units for the concentration, advisory information, or combinations thereof.

For total parenteral nutrition (TPN) delivery, the limits included in each subdirectory 42 include a hard rate limit, a soft rate limit, a hard dose limit (e.g., hard dose rate limit and hard dose vtbi limit), a soft dose limit (e.g., soft dose rate limit and soft dose vtbi limit), units for time, a concentration of the drug, units for the concentration, advisory information, or combinations thereof.

For patient controlled analgesia (PCA) delivery, the limits included in each subdirectory 42 include a maximum loading dose, a loading dose delivery rate, a maximum bolus size, a bolus delivery rate, a minimum bolus lockout time, a maximum bolus lockout time, a maximum basal rate, advisory information, or combinations thereof.

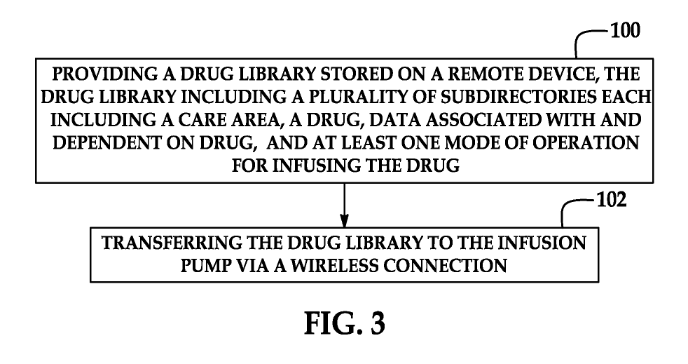

An example of the method of loading the drug library 40 into the infusion pump 12 is schematically depicted in FIG. 3. The method includes providing the drug library 40 (as shown by reference numeral 100) and transferring the drug library 40 to the infusion pump 12 via a wireless connection (as shown by reference numeral 102). It is to be understood that when the drug library 40 is transferred to the infusion pump 12, the entire master drug library 40 is transferred.

In an embodiment, prior to transferring the drug library 40 to the infusion pump 12, the remote device 22 including the drug library 40 stored thereon is wirelessly connected to the infusion pump 12 via the wireless connection. Thereafter, the remote device 22 confirms that the infusion pump 12 is in fact authorized and allowed to receive the drug library 40 download. In an example, confirming may be accomplished by obtaining a serial number or other identification number for a particular infusion pump 12 and checking the number against a master list of authorized pumps. If the number identifying the infusion pump 12 matches an identification number on the list, the pump 12 is considered to be an authorized pump and the remote device 22 is allowed to transfer the entire drug library to the infusion pump 12.

In another embodiment, for relatively safe transfer of the drug library 40 from the remote device 22 to the infusion pump 12, the drug library 40 may be encoded and the encoded drug library is transferred to the infusion pump 12. The infusion pump 12 may be equipped with suitable decoding algorithms, operating via a processor (not shown) internal to the pump 12, for decoding the encoded drug library upon receiving it from the remote device 22. The entire, decoded drug library 40 is then stored in the memory 14 of the infusion pump 12.

Prior to using the drug library 40, the infusion pump 12 checks the transferred or transferred and decoded version of the drug library 40 for any errors. Checking may be accomplished using, e.g., cyclic redundancy checks (CRC). The error check may also involve a check for over-the-air transmission problems or errors. If, for example, an error is found, the pump 12 requests a re-transmission of the drug library from the remote device 22. If, on the other hand, an error is not found, an indication of the same will appear on, e.g., the display 20 of the pump 12 and infusion of the drug may commence.

In yet another embodiment, at least one pump setting may be wirelessly transmitted or otherwise transferred from the remote device 22 to the pump 12. Transferring of the pump settings may be accomplished in addition to, or separate from transferring the drug library 40. The pump settings are generally used to enable customization of the pump 12 for a care area. Examples of possible pump settings are summarized as follows:

Enable/disable a default flow rate setting;

Enable/disable a default priming rate setting;

Enable/disable a setting requiring the entry of caregiver identification;

Enable/disable a setting requiring the entry of patient identification;

Limit maximum and minimum bolus dose lockout time, bolus dose, loading dose rate, and bolus per hour;

Limit basal rate and bolus dose rate;

Enable/disable low power display and low power motor;

Enable/disable continuous delivery mode, dose-based deliver mode, intermittent delivery mode, PCA delivery mode, TPN delivery mode;

Enable/disable intravenous delivery and epidural delivery;

Enable/disable password screen and screen lock;

Enable/disable exceeding a soft limit;

Set volume level; and

Enable/disable air-in-line trigger, occlusion trigger, and occlusion retries.

While several embodiments have been described in detail, it will be apparent to those skilled in the art that the disclosed embodiments may be modified. Therefore, the foregoing description is to be considered exemplary rather than limiting.

What is claimed is:

1. A method of loading a drug library into an infusion pump, the method comprising:
providing a drug library stored on a remote device, the drug library including a plurality of subdirectories, each subdirectory including a care area, at least one drug, data associated with and dependent upon the at least one drug, and at least one mode of operation for infusing the at least one drug; and
transferring the drug library to the infusion pump via a wireless connection.

2. The method as defined in claim 1 wherein the remote device is a computer.

3. The method as defined in claim 1 wherein the data associated with and dependent upon the at least one drug includes a plurality of limits for infusing the drug based at least on a name of the at least one drug, the care area associated with the drug name, the at least one mode of operation, or combinations thereof.

4. The method as defined in claim 3 wherein the at least one mode of operation is a continuous delivery, and wherein the plurality of limits includes a hard rate limit, a soft rate limit, a hard dose limit, a soft dose limit, units for time, a concentration of the at least one drug, units for the concentration, advisory information, or combinations thereof.

5. The method as defined in claim 3 wherein the at least one mode of operation is a weight-based continuous delivery, and wherein the plurality of limits includes a hard rate limit, a soft rate limit, a hard dose limit, a soft dose limit, units for time, a concentration of the at least one drug, units for the concentration, advisory information, or combinations thereof.

6. The method as defined in claim 3 wherein the at least one mode of operation is a delivery based on a dose, and wherein the plurality of limits includes a hard rate limit, a soft rate limit, a hard dose limit, a soft dose limit, units for time, a minimum delay time, a concentration of the drug, units for the concentration, advisory information, or combinations thereof.

7. The method as defined in claim 3 wherein the at least one mode of operation is a total parenteral nutrition delivery, and wherein the plurality of limits includes a hard rate limit, a soft rate limit, a hard dose limit, a soft dose limit, units for time, a concentration of the drug, units for the concentration, advisory information, or combinations thereof.

8. The method as defined in claim 3 wherein the at least one mode of operation is a patient controlled analgesia mode, and wherein the plurality of limits includes a maximum loading dose, a loading dose delivery rate, a maximum bolus size, a bolus delivery rate, a minimum bolus lockout time, a maximum bolus lockout time, a maximum basal rate, advisory information, or combinations thereof.

9. The method as defined in claim 1 wherein prior to transferring the drug library to the infusion pump, the method further comprises:
wirelessly connecting the remote device to the infusion pump; and
confirming that the infusion pump is allowed to receive the drug library.

10. The method as defined in claim 1 wherein when the drug library is transferred from the remote device to the infusion pump, the method further comprises:
encoding the drug library;
transmitting the encoded drug library to the infusion pump;
at the infusion pump, decoding the encoded drug library; and
storing the drug library into a memory device associated with the infusion pump.

11. The method as defined in claim 10, further comprising:
checking, via the infusion pump, the decoded drug library for errors; and
when an error is found, requesting a re-transmission of the drug library from the remote device.

12. The method as defined in claim 1, further comprising wirelessly transmitting at least one pump setting for enabling customization of an infusion process.

13. A drug library transferred from a remote device to an infusion pump, the drug library comprising a plurality of subdirectories, each of the subdirectories including a care area, at least one drug, data associated with and dependent on the at least one drug, and at least one mode of operation for infusing the at least one drug.

14. The drug library as defined in claim 13 wherein the data associated with and dependent upon the at least one drug includes a plurality of limits for infusing the drug based at least on a name of the at least one drug, the care area associated with the drug name, the at least one mode of operation, or combinations thereof.

15. The drug library as defined in claim 13 wherein the at least one mode of operation is a continuous delivery, and wherein the plurality of limits includes a hard rate limit, a soft rate limit, a hard dose limit, a soft dose limit, units for time, a concentration of the drug, units for the concentration, advisory information, or combinations thereof.

16. The drug library as defined in claim 13 wherein the at least one mode of operation is a weight-based continuous delivery, and wherein the plurality of limits includes a hard rate limit, a soft rate limit, a hard dose limit, a soft dose limit, units for time, a concentration of the drug, units for the concentration, advisory information, or combinations thereof.

17. The drug library as defined in claim 13 wherein the at least one mode of operation is a delivery based on a dose, and wherein the plurality of limits includes a hard rate limit, a soft rate limit, a hard dose limit, a soft dose limit, units for time, a minimum delay time, a concentration of the drug, units for the concentration, advisory information, or combinations thereof.

18. The drug library as defined in claim 13 wherein the at least one mode of operation is a total parenteral nutrition delivery, and wherein the plurality of limits includes a hard rate limit, a soft rate limit, a hard dose limit, a soft dose limit, units for time, a concentration of the drug, units for the concentration, advisory information, or combinations thereof.

19. The drug library as defined in claim 13 wherein the at least one mode of operation is a patient controlled analgesia mode, and wherein the plurality of limits includes a maximum loading dose, a loading dose delivery rate, a maximum bolus size, a bolus delivery rate, a minimum bolus lockout time, a maximum bolus lockout time, a maximum basal rate, advisory information, or combinations thereof.

20. An infusion pump system, comprising:
an infusion pump;
a computer, remote from the infusion pump, having a drug library stored therein, the drug library including a plurality of subdirectories, each subdirectory including a care area, at least one drug, data associated with and dependent upon the at least one drug, and at least one mode of operation for infusing the at least one drug; and
a wireless connection established between the computer and the infusion pump, the wireless connection enabling the computer to transfer the drug library to the infusion pump.
